(19) United States
(12) Patent Application Publication
(10) Pub. No.: US 2018/0271809 A1
(43) Pub. Date: Sep. 27, 2018

(54) USE OF NICLOSAMIDE IN THE TREATMENT OF P53-DEFICIENT CELLS

(71) Applicants: AGENCY FOR SCIENCE, TECHNOLOGY AND RESEARCH, Singapore (SG); IFOM, THE FIRC INSTITUTE OF MOLECULAR ONCOLOGY FOUNDATION, Milan (IT)

(72) Inventors: Chit Fang CHEOK, Singapore (SG); Ramesh KUMAR, Milan (IT)

(21) Appl. No.: 15/761,081

(22) PCT Filed: Sep. 16, 2016
(86) PCT No.: PCT/SG2016/050453

§ 371 Date: Mar. 16, 2018

(30) Foreign Application Priority Data
Sep. 16, 2015 (SG) 10201507716Q

(51) Int. Cl. A61K 31/167; A61K 45/06; G01N 33/50; A61P 35/00

(52) U.S. Cl. CPC A61K 31/167; A61P 35/00; G01N 33/5011; A61K 45/06

(57) ABSTRACT

The present disclosure describes a method of treating p53-deficient cells using a niclosamide derivative according to formula (I). The present disclosure further describes a method of treating cancer using the compound according to formula I, as well as methods of screening to identify a compound that selectively target cells wherein p53 is deficient or non-functional.

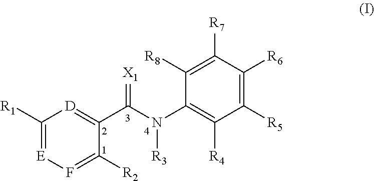

### CROSS-REFERENCE TO RELATED APPLICATIONS

This application claims the benefit of priority of Singapore provisional application No. 10201507716Q, filed 16 Sep. 2015, the contents of it being hereby incorporated by reference in its entirety for all purposes.

### FIELD OF THE INVENTION

The present invention relates generally to the field of molecular biology. In particular, the present invention relates to compounds for the treatment of p53-deficient cells.

### BACKGROUND OF THE INVENTION

TP53, also more commonly known as p53, is one of the most commonly mutated tumour suppressor genes in human cancer. As known in the art, p53 functions as a node in numerous signalling pathways in that many important biological activities are regulated by the transcriptional activity of the p53 gene from fertility and development to maintaining genomic stability and cell death.

Studies have shown that p53 is part of a family that can be subject to alternative splicing. The fact that p53 was originally described as an oncogene has come full circle with mutant p53 having been shown to exhibit gain of function properties that actually drive tumour progression and metastasis. p53 is therefore of therapeutic importance and numerous strategies are being employed to reconstitute its expression in tumours. While much has been known about the biological activity and functions of p53, to date, a strategy to target cells with mutant p53 or loss of p53 functions is still absent. Given the high frequency of p53 mutations in tumours, there is therefore a need to find a therapeutic approach to target and treat tumours which show a loss of p53 functions.

### SUMMARY OF THE INVENTION

In one aspect, the present invention refers to a method of treating cancer using a compound as defined in formula I:

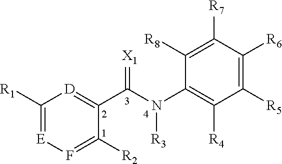

or derivative thereof, wherein D is N or CR⁹; E is N or CR¹⁰; F is N or CR¹¹; R¹ is H, halide, OR¹², SR¹³, NR¹⁴, R¹⁵,

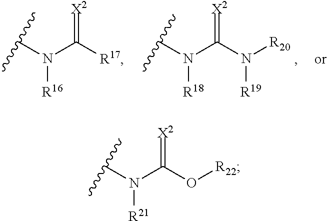

R² is H, OH or OR¹²; R³ is H, C₁₋₇ alkyl, C₂₋₇ alkenyl, C₂₋₇ alkynyl, C₂₋₆ heterocyclyl; C₆₋₁₂ aryl, C₇₋₁₄ alkaryl; C₃₋₁₀ alkheterocyclyl, or C₁₋₇ heteroalkyl; or R² and R³ combine to form a six-membered ring in which position 1 is connected to position 4 by

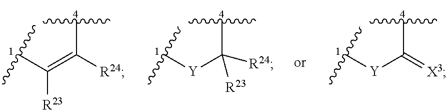

R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl; R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰,

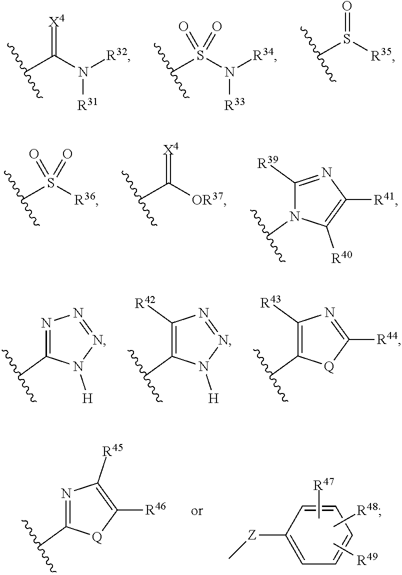

each X¹, X², X³, and X⁴ is, independently, O, S; or NR³⁸; Y is CR²⁵R²⁶, O, S, or NR²⁷; Z is O, S, or CR⁵⁰R⁵¹; each Q is, independently, O, S, or NR⁵² ; R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO_{2;} R¹² and R¹³ are each, independently, acyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, wherein the cancer comprises p53-deficient cells or cells wherein p53 is non-functional.

In another aspect, the present invention refers to a method of treating cancer using the compound as defined herein, wherein the cancer comprises p53-deficient cells or cells wherein p53 is non-functional.

In yet another aspect, the present invention refers to a method of identifying whether a patient is likely or unlikely to respond to a treatment, wherein the treatment comprises the administration of the compound as defined herein, wherein the method comprising determining whether a cell or tissue sample obtained from a patient comprises a) p53-positive or p53-deficient cells or b) p53 that is non-functional, wherein the identification of the cell or tissue sample as p53-deficient or the cells or tissue sample containing p53 that is non-functional identifies a patient that is likely to respond to the treatment, wherein the identification of said sample as p53-positive identifies a patient that is unlikely to respond to the treatment.

In another aspect, the present invention refers to use of the compound as defined herein, or derivatives thereof, in the manufacture of a medicament for treating cancer, wherein the cancer comprises p53-deficient cells or cells wherein p53 is non-functional.

In yet another aspect, the present invention refers to a method of screening to identify a compound that selectively targets a first cell type or a second cell type, wherein the first cell type is a p53-deficient cell or a cell wherein p53 is non-functional, and wherein the second cell type is a p53-positive cell, and wherein the first cell type is labelled with a first detectable marker and wherein the second cell type is labelled with a second detectable marker, the method comprising: A. contacting the first and second cell types with the compound; and B. determining the relative amounts of the first and second detectable markers; wherein the first and second detectable marker are independently detectable and wherein a relative increase in the amount of the first marker in comparison to the amount of the second marker is indicative of a compound selectively targeting a p53-positive cell and wherein a relative increase in the amount of the second marker is indicative of a compound selectively targeting a p53-deficient cell or a cell wherein p53 is non-functional.

In a further aspect, the present invention refers to a kit comprising the compound as described herein and a further therapeutic agent.

### BRIEF DESCRIPTION OF THE DRAWINGS

The invention will be better understood with reference to the detailed description when considered in conjunction with the non-limiting examples and the accompanying drawings, in which:

FIG. 1 shows fluorescent images of cells using an In Cell Analyzer 2000. Fluorescent images of H2B-GFP labelled HCT116 p53^{+/+} and H2BRFP labelled HCT116 p53^{-/-} cells are captured and cell numbers are quantified. These images (singular and merged) show the specific labelling of the cells according to the presence or absence of p53.

FIG. 2 shows a combined column and line graph in FIG. 2A (top panel), a line graph depicting the influence of niclosamide on cell viability (FIG. 2B) and a micrograph image of crystal violet stained cells in FIG. 2C and 2D (bottom panel). FIG. 2A shows cells imaged using an In Cell Analyzer 2000. Co-cultures of H2B-GFP labelled HCT116 p53^{+/+} and H2B-RFP labelled HCT116 p53^{-/-} cells are treated with the indicated concentrations of niclosamide for 48 hours. Cells were left to recover for 7 days before imaging. Relative percentages of H2B-GFP labelled HCT116 p53^{+/+} and H2B-RFP labelled are quantified and represented in a graph. FIG. 2C and 2D: HCT 116 p53 mutant and wild type on its own (2C) or HCT116 p53^{+/+} are transfected either with Non Targeting (NT) shRNA, or p53 specific shRNAs (p53 sh I and p53 sh II). Cells were treated with the indicated concentrations of niclosamide for 48 hours and recovered in fresh media for 12 days. FIG. 2C and 2D shows an image of colonies of cells in cell culture plates stained with crystal violet. Crystal violet only stains cells that were alive at the time of staining. Therefore, increased de-staining is indicative of cell death. This data shows the effect of niclosamide treatment on the viability of HCT166 cells, in particular HCT116 p53^{-/-} cells.

FIG. 3 shows the experimental data gathered in determining the effect of niclosamide on apoptosis behaviour of p53-deficient human fibroblast cells compared to wild type human fibroblasts. FIG. 3A shows bright-field images of normal primary human fibroblast cells are imaged 7 days after cells were treated with niclosamide. Shcontrol, shp53 (III) and shp53 (II) refers to cells transfected with vectors encoding shRNA control and shRNAs to p53. FIG. 3B shows multiple flow cytometry graphs depicting the data from an Annexin V-FITC assay. Annexin V-FITC assay was performed on normal human fibroblast cells transfected with shRNA control or p53-specific shRNA. Cells were treated with indicated concentrations of niclosamide (1-4 μM) and recovered in fresh media 48 hours later. 7 days later, cells were stained using Annexin-V FITC and percentages of Annexin-V FITC positive populations were quantified using flow cytometry. Taken together, the data shown in FIG. 3 show that p53-deficient cells have an increase sensitivity/susceptibility to niclosamide treatment, which results in the preferential killing of p53 deficient cells.

FIG. 4 shows the results of testing of mouse embryonic fibroblast (MEF) cells with either wild type p53 or harbouring p53 mutations (+/R172 or R172/R172) for niclosamide sensitivity. FIG. 4A shows bright filed image data, which suggests that unlike wild type cells, p53 mutant cells are sensitive to niclosamide. Like human fibroblast cells, these cells were also tested for apoptotic cell death, the data of which is shown in the graphs shown in FIG. 4B). Images of crystal violet staining of the cells indicated that p53 mutant MEF cells are sensitive to niclosamide, as shown in FIG. 4C). Both HCT116 cells with wild type p53 and those which were p53 deficient were treated with niclosamide, and protein samples were immunoblotted with Caspase-3 antibody. A clear induction of the cleaved caspase in p53 minus (that is p53 deficient) cells indicates the apoptotic cells death in those cells. This data is shown represented in the image of a western blot as shown in FIG. 4D.

FIG. 5 shows fluorescent microscopy captures images of HCT116 p53^{+/+} (green fluorescent protein (GFP) labelled) cells and HCT116 p53^{-/-} (red fluorescent protein (RFP) labelled) cells grown in co-culture and incubated with the indicated concentrations of niclosamide. The data shown in FIG. 5 shows the capability of niclosamide to preferentially reduce the spheroid growth of p53-deficient cells.

FIG. 6 shows the set up and data gathered in an in vivo murine tumour model for testing the use of niclosamide in treating p53^{-/-} tumour cells. FIG. 6A shows a schematic layout of the experiment. FIG. 6B shows a picture of a nude mice dissected to reveal tumour growth on the right flank (R: DMSO treated control cells (negative control)) but to a lesser extent on the left flank (L: cells treated with niclosamide). FIG. 6C shows a picture of the resected tumours, which were weighed, the measurements of which are displayed in a graph on the right. R: Right flank: DMSO treated control cells (negative controls). L: Left flank: cells treated with niclosamide. The data shown in FIG. 6 shows that niclosamide has an anti-tumour effect in vivo, that is that the treatment of tumours with niclosamide results in significant growth reduction in tumour volume in vivo.

### Definitions

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7, 1 to 2, 1 to 3, 1 to 4, 1 to 5, 1 to 6, 2 to 3, 2 to 4, 2 to 5, 2 to 6, 2 to 7, 3 to 4, 3 to 5, 3 to 6, 3 to 7, 4 to 5, 4 to 6, 4 to 7, 5 to 6, 5 to 7, 6 to 7 carbon atoms includes each of C₁, C₂, C₃, C₄, C₅, C₆, and C₇. A C₁₋₇heteroalkyl, for example, includes from 1 to 6, 1 to 2, 1 to 3, 1 to 4, 1 to 5, 2 to 3, 2 to 4, 2 to 5, 2 to 6, 3 to 4, 3 to 5, 3 to 6, 4 to 5, 4 to 6, 5 to 6 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of acyclic and cyclic groups, i.e., cycloalkyl. Cyclic groups can be monocyclic or polycyclic and can have from 3 to 6 ring carbon atoms, inclusive. Exemplary cyclic groups include cyclopropyl, cyclobutyl, cyclopentyl, and cyclohexyl groups. For example, the C₁₋₇ alkyl group may be substituted or unsubstituted. Exemplary substituents include, but are not limited to alkoxy; aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₇ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

As used herein, the term “C₂₋₇ alkenyl” refers to a branched or unbranched hydrocarbon group containing one or more double bonds and having from 2 to 7 carbon atoms. A C₂₋₇ alkenyl may optionally include acyclic, monocyclic or polycyclic rings, in which each ring desirably has from three to six members. The C₂₋₇ alkenyl group may be substituted or unsubstituted. Exemplary substituents include, but are not limited to alkoxy, aryloxy, sulfhydryl, alkylthio, arylthio, halide, hydroxyl, fluoroalkyl, perfluoralkyl, amino, aminoalkyl, disubstituted amino, quaternary amino,hydroxyalkyl, carboxyalkyl, and carboxyl groups. C₂₋₇ alkenyls include, without limitation, vinyl; allyl; 2-cyclopropyl-i-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3 -dimethyl-3-butenyl; 1,3-dimethyl-3-butenyl; 1,1-dimethyl-3-butenyl and 2,2-dimethyl-3-butenyl.

As used herein, the term “C₂₋₇ alkynyl” refers to a branched or unbranched hydrocarbon group containing one or more triple bonds and having from 2 to 7 carbon atoms. A C₂₋₇ alkynyl may optionally include acyclic, monocyclic, bicyclic, or tricyclic rings, in which each ring desirably has five or six members. The C₂₋₇ alkynyl group may be substituted or unsubstituted. Exemplary substituents include, but are not limited to, alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl ; 1-methyl-2-propynyl; 1-methyl-2-butynyl ; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

As used herein, the term “C₂₋₆ heterocyclyl” refers to a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring.

The heterocyclyl group may be substituted or unsubstituted. Exemplary substituents include, but are not limited to, alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulphur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. In one example, when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidinyl, 4aH-carbazole, 4H-quinolizinyl, 6H- 1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolrnyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylpyrimidinyl, phenanthridinyl, phenanthrolinyl, phenarsazinyl, phenazinyl, phenothiazinyl, phenoxathionyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyridyl, pyrimidinyl, pyrrolidinyl, pyrrolinyl, pyrrolyl, quinazolinyl, quinolinyl, 4H-quinolizinyl, quinoxahnyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. In one example, 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. In one example, 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

As used herein, the term “C₆₋₁₂ aryl” refers to an aromatic group having a ring system comprised of carbon atoms with conjugated 7C electrons (e.g., phenyl). An aryl group can have between 6 to 12 carbon atoms. Aryl groups may optionally include monocyclic, bicyclic, or tricyclic rings, in which each ring desirably has five or six members. The aryl group may be substituted or unsubstituted. Exemplary substituents include, but are not limited to alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

As used herein, the term “C₇₋₁₄ alkaryl” refers to an alkyl substituted by an aryl group (e.g., benzyl, phenethyl, or 3,4-dichlorophenethyl) having from 7 to 14 carbon atoms.

As used herein, the term “C₃₋₁₀ alkheterocyclyl” refers to an alkyl substituted heterocyclic group having from 3 to 10 carbon atoms in addition to one or more heteroatoms (for example, but not limited to, 3-furanylmethyl, 2-furanylmethyl, 3-tetrahydrofuranylmethyl, or 2-tetrahydrofuranylmethyl).

As used herein, the term “C₁₋₇ heteroalkyl” refers to a branched or unbranched alkyl, alkenyl, or alkynyl group having from 1 to 7 carbon atoms in addition to one or more heteroatoms, wherein one or more methylenes (CH₂) or methines (CH) are replaced by nitrogen, oxygen, sulfur, carbonyl, thiocarbonyl, phosphoryl, or sulfonyl.

Heteroalkyls include, without limitation, tertiary amines, secondary amines, ethers, thioethers, amides, thioamides, carbamates, thiocarbamates, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include, but are not limited to alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

As used herein, the term “acyl” refers to a chemical moiety with the formula R—C(O)—, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

As used herein, the term “halide” refers to bromine, chlorine, iodine, or fluorine.

As used herein, the term “fluoroalkyl” refers to an alkyl group that is substituted with a fluorine.

As used herein, the term “perfluoroalkyl” refers to an alkyl group consisting of only carbon and fluorine atoms.

As used herein, the term “carboxyalkyl” refers to a chemical moiety with the formula —(R)—COOH, wherein R can be, but is not limited to, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

As used herein, the term “hydroxyalkyl” refers to a chemical moiety with the formula —(R)—OH, wherein R can be, but is not limited to C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

As used herein, the term “alkoxy” refers to a chemical substituent of the formula —OR, wherein R can be, but is not limited to C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

As used herein, the term “aryloxy” refers to a chemical substituent of the formula —OR, wherein R is, for example, a C₆₋₁₂ aryl group.

As used herein, the term “alkylthio” refers to a chemical substituent of the formula —SR, wherein R can be, but is not limited to, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

As used herein, the term “arylthio” refers to a chemical substituent of the formula —SR, wherein R is, for example, a C₆₋₁₂ aryl group.

As used herein, the term “quaternary amino” refers to a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

As used herein, the term “mutation” refers to a sudden random change in the genetic material of a cell that potentially can cause it and all cells derived from it to differ in appearance and behaviour (i.e. in phenotype) from the normal, unmutated type. An organism affected by a mutation (especially one with visual effects) is described as a mutant. Somatic mutations affected the non-reproductive cells and are therefore restricted to the tissues of a single organism, whereas germ-line mutations, which occur in the reproductive cells or their precursors, may be transmitted to the organism's descendants and cause abnormal development. In the present application, a mutation refers to the permanent alteration of the nucleotide sequence of the genome of an organism, virus, or extra-chromosomal DNA or other genetic elements. Mutations result from damage to DNA, which then may undergo error-prone repair, or else may cause an error during replication. Mutations may also result from insertion or deletion of segments of DNA due to mobile genetic elements. Different types of mutations are, but are not limited to, substitution mutations, single base substitutions, silent mutations, missense mutations, nonsense mutations, neutral mutations, insertions, deletions, amplifications, chromosomal translocations, interstitial deletions, and chromosomal inversions. Mutations can also be classified according to their result, that is, for example, a loss of function mutation (also known as an inactivating mutation) results in a gene product having none or little function, compared to the unmutated version of the gene product. A gain of function mutations (also known as an activating mutation) therefore results in an enhancement of the unmutated gene product's properties.

As used herein, the term “polymorphism” refers to a variation or difference in the sequence of a genetic region that arises in some of the members of a species. Variant sequences can be defined with reference to an arbitrary or non-arbitrary standard sequence for the species. A polymorphism is thus said to be “allelic,” in that, due to the existence of the polymorphism, some members of a species may have the “standard” sequence (i.e. the standard “allele”) whereas other members may have a variant sequence (i.e., a variant “allele”). Thus, as used herein, an allele is one of two or more alternative versions of a gene or other genetic region at a particular location on a chromosome. In the simplest case, only one variant sequence may exist, and the polymorphism is thus said to be bi-allelic. In other cases, the species' population may contain multiple alleles, and the polymorphism is termed tri-allelic, etc. A single gene or genetic region may have multiple different unrelated polymorphisms. For example, it may have one bi-allelic polymorphism at one site, another bi-allelic polymorphism at another site and a multi-allelic polymorphism at yet another site. When all the sequences for a group of alleles at a chromosomal locus in a plant are the same, the alleles are said to be “homozygous” at that locus. When the sequence of any allele at a particular locus in a plant is different, the population of alleles is said to be “heterozygous” at that locus. Phenotypic traits can vary due to environmental and/or genetic factors. For example, polymorphisms at a particular chromosomal locus can affect the phenotypic trait associated with that locus.

As used herein, the term “derivative” refers to is a compound that is derived from a similar compound by a chemical reaction. A derivative may also be known as a structural or functional analogue.

In the present specification, preventing a disease refers to inhibiting completely, or in part, the development or progression of a disease, for example in a person who is known to have a predisposition to a disease. An example of a person with a known predisposition is someone with a history of cancer in the family, or who has been exposed to factors that predispose the subject to the development of a tumour.

Also, in the present specification, treating a disease refers to a therapeutic intervention that inhibits, or suppressed the growth of a tumour, eliminates a tumour, ameliorates at least one sign or symptom of a disease or pathological condition, or interferes with a pathophysiological process, after the disease or pathological condition has begun to develop.

As used herein, the term “synthetically lethal” refers to the scenario in which a combination of mutations in two or more genes leads to cell death, that is to say the culmination of the mutations in these two of more genes is lethal to the cells. However, in the case of mutations being synthetically lethal, the occurrence of each mutation alone does not result in cell death. For example, in regards to two genes in a host, gene A and gene B, if a mutation being present in each of genes A and B results in cell death, then the mutation is considered to be synthetically lethal. The presence of an unmutated copy of gene A and only a mutated copy of gene B (or vice versa, that is a mutated copy of gene A and an unmutated copy of gene B) in the cell is not lethal to said cell. In such a scenario (that is for example, when an unmutated version of gene A is present with a mutated version of gene B), this can result in a synthetic sickness, which is when the combination of mutations (also termed genetic events) results in a non-lethal reduction of fitness in a host. Thus, synthetic lethality is a result of the capability of organisms to maintain errors for margins and/or buffer schemes which in turn allow for phenotypic stability despite genetic variation, environmental changes and random events such as mutations. All these factors play a part in the overall (genetic) robustness of an organism.

### DETAILED DESCRIPTION OF THE PRESENT INVENTION

The approach taken was to perform a throughput screen to discover molecules which are synthetically lethal to cells having a loss of p53 functions. This throughput screening was performed using a cell-based competition assay comprising of HCT116 wild type and HCT116 p53^{-/-} cells, which were fluorescent labelled. The pharmakon library, consisting of more than 1600 compounds, was screened for the compounds' differing activity on the viability of HCT116 p53^{-/-} cells. Secondary screens were performed to validate the candidate compounds, which were selected on the basis of their preferential killing of HCT116 p53^{-/-} cells.

In an independent drug screen using the so-called phamarkon library from MicroSource Discovery Systems, comprising of 1600 different FDA approved compounds, it was shown that compounds according to the compound as described herein, for example niclosamide and derivatives thereof, are effective at killing p53-deficient cells. The efficacy of a compound in killing and/or treating a certain disease, for example cancer, can be determined by comparing the effect of said compound on diseased cells and the effect of said compound on disease-free cells. The diseased cells may be obtained from a patient or a subject suffering from the disease, but may also be specific (diseased) cell lines in cell culture. While it is possible to tell a diseased cell from a non-diseased cell based on its phenotype in, for example microscopic analysis, it is also possible to label the different types of cells each with a different marker, and then use those markers in the differential analysis of the resulting cell population after treatment with the compound in question. Therefore, the increase and/or decrease of a labelling signal of one cell type over the other is indicative of the strength in terms of efficacy (when looking at the time and dose required), as well as lethality of the compound to be analysed. Thus, in one example disclosed herein is a method of screening to identify a compound that selectively targets a first cell type or a second cell type, wherein the first cell type is a p53-deficient cell or a cell wherein p53 is non-functional and wherein the second cell type is a p53-positive cell, and wherein the first cell type is labelled with a first detectable marker and wherein the second cell type is labelled with a second detectable marker, the method comprising (a) contacting the first and second cell types with the compound; and (b) determining the relative amounts of the first and second detectable markers; wherein the first and second detectable marker are independently detectable and wherein a relative increase in the amount of the first marker in comparison to the amount of the second marker is indicative of a compound selectively targeting a p53-positive cell and wherein a relative increase in the amount of the second marker is indicative of a compound selectively targeting a p53-deficient cell or a cell wherein p53 is non-functional. In one example, the detectable marker is, but is not limited to, a fluorescent compound, a radioisotope compound, a non-radioisotope compound, a bioluminescent compound, a chemiluminescent compound, a metal chelator compound, a chromogenic compound, an X-radiographic compound, and an enzyme. In another example, the fluorescent compound is but is not limited to, a Green Fluorescent Protein (GFP), Red Fluorescent Protein (RFP), fluorescein, rhodamine, phycoerytherin, phycocyanin, allophycocyanin, o-phthaldehyde and fluorescamine.

In another example, the first cell type is labelled with Red Fluorescent Protein (RFP) and the second cell type is labelled with Green Fluorescent Protein (GFP). In another example, the cell types are contacted with the compound in vitro, ex vivo or in vivo. In another example, the relative amounts of the first and second marker are determined using fluorescence-activated cell sorting (FACS) or quantitative imaging microscopy.

p53 is known in the art as a tumour suppressor gene, meaning that the activity of this gene and the protein(s) that it encodes stops the formation of tumours in a host. Cellular mechanisms such as transcription regulation, activation of DNA repair, arrest of cell cycle and initiation of apoptosis in irreparable cells have been attributed to the p53 gene's anti-cancer characteristics.

For example, the compound niclosamide was found to activate apoptosis in p53-deficient cells to a greater extent than that observed in p53 wild type cells. This provides a function of niclosamide as a drug in targeting p53-deficient cells. Of note, p53 deficiency is found in more than 50% of all tumours, thereby indicating that niclosamide targets cellular pathways that, when inhibited, are synthetically lethal for the p53 deficient cell. Therefore, in one example, the compound claimed herein can be used in the selective killing of p53-deficient cells. In another example, disclosed herein is the use of a single compound in targeting p53-deficient tumour cells in an anti-cancer treatment. In another example, the compound is niclosamide, or a derivative thereof. In yet another example, a method of treating cancer using the compound as described herein is disclosed, wherein the cancer comprises p53-deficient cells or cells wherein p53 is non-functional In another example, a method of treating cancer is disclosed using 2′,5-dichloro-4′-nitrosalicylanilide (niclosamide) or derivative thereof as defined herein, wherein the cancer comprises p53-deficient cells or cells wherein p53 is non-functional. In another example, use of the compound as disclosed herein, or derivatives thereof, in the manufacture of a medicament for treating cancer, wherein the cancer comprises p53-deficient cells or cells wherein p53 is non-functional is disclosed. In any of the examples disclosed herein, the compound is 2′,5-dichloro-4′-nitrosalicylanilide (niclosamide) or derivative thereof.

Also disclosed herein are methods of determining a subject's susceptibility to a treatment with a drug that targets p53-deficient cells. This is done by, for example, comparing the presence or absence of a particular gene in a sample obtained from a diseased subject and comparing the levels of said analysed gene with the levels of the same gene determined in a sample taken from a subject who is not suffering from that disease (that is, the subject is considered to be healthy). This determination can also be performed on the translation and/or transcription products of said target gene, for example, but not limited to, mRNA transcripts, RNA transcripts, proteins, isomers and variations thereof. In another example, the gene can be a gene playing a role in the same pathway as p53. In one example, the gene is p53. In another example, the determination is performed on the transcription product of p53, that is on a protein encoded by p53. Thus, in one example, a method of identifying whether a cancer patient is likely or unlikely to respond to a treatment is disclosed herein, wherein the treatment comprises the administration of the compound as disclosed herein or derivative thereof as defined herein, wherein the method comprising determining whether a cell or tissue sample from a patient comprises a) p53-positive or p53-deficient cells or b) p53 that is non-functional, wherein the identification of the cell or tissue sample as p53-deficient or the cells or tissue sample containing p53 that is non-functional identifies the patient as likely to respond to the treatment, wherein the identification of said sample as p53-positive identifies the patient as unlikely to respond to the treatment. In one example, the compound is 2′,5-dichloro-4′-nitrosalicylanilide (niclosamide) or derivatives thereof.

Mutations or deletions of the p53 gene are known to cause various diseases, for example cancer. In one example, the cancer can be, but is not limited to, breast, brain, head and neck, lung, cervix, liver, kidney, cells of the blood (for example leukemia), prostate, colon, rectum, colorectal, renal, liver, intestines, skin, epithelium, bone, pancreas, lymphoma, ovarian, rectal, sarcoma, testicular, uterine and cartilage. In another example, the cancer is, but is not limited to melanomas, myelomas, carcinomas, sarcomas, lymphomas, blastomas and germ cell tumours. In yet another example, the cancer is, but is not limited to leukemia, lymphoma, myeloma, skin cancer such as basal cell carcinoma (BCC), squamous cell carcinoma (SCC) or melanoma, breast cancer, head and neck cancer, such as brain cancer, colorectal cancer, colon cancer, rectal cancer, lung cancer, such as non-small cell lung cancer (NSCLC), ovarian cancer, renal cancer, prostate cancer, liver cancer, and human papilloma virus (HPV) -associated cancer such as cervical cancer.

In one example, there is disclosed herein a method of treating cancer using a compound as defined in formula I:

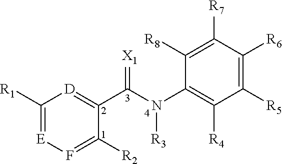

or derivative thereof, wherein
D is N or CR⁹; E is N or CR¹⁰; F is N or CR¹¹;
R¹ is H, halide, OR¹², SR¹³, NR¹⁴, R¹⁵,

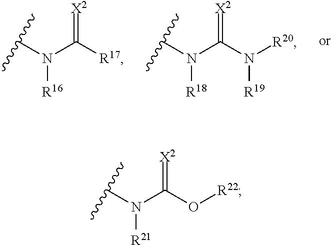

R² is H, OH or OR¹²;
R³ is H, C₁₋₇ alkyl, C₂₋₇ alkenyl, C₂₋₇ alkynyl, C₂₋₆ heterocyclyl; C₆₋₁₂ aryl, C₇₋₁₄ alkaryl; C₃₋₁₀ alkheterocyclyl, or C₁₋₇ heteroalkyl; or R² and R³ combine to form a six-membered ring in which position 1 is connected to position 4 by

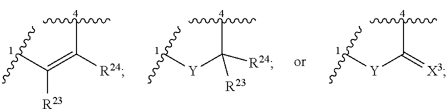

R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰,

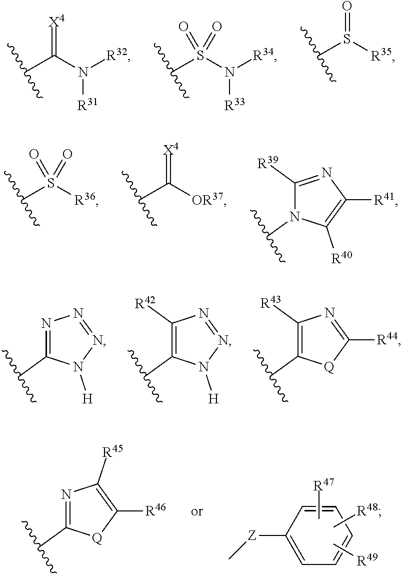

each X¹, X², X³, and X⁴ is, independently, O, S; or NR³⁸;
Y is CR²⁵R²⁶, O, S, or NR²⁷;
Z is O, S, or CR⁵⁰R⁵¹;
each Q is, independently, O, S, or NR⁵²;
R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO_{2;}
R¹² and R¹³ are each, independently, acyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R³⁹R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, wherein the cancer comprises p53-deficient cells or cells wherein p53 is non-functional. In one example, X₁ is an oxygen atom, R² is OH and R³ is H. In another example, D, E and F are each independently CH. In another example, R¹ is H or halide. In another example, R¹ is Cl. In one example, R⁴ and R⁸ are independently H, halide or CF₃. In another example, R⁴ is Cl. In another example, R⁸ is H. In one example, R⁵, R⁶ and R⁷ are independently H, halide, NO₂, CO₂H, SO₃H, CF₃ or CN. In one example, R⁵ is H. In another example, R⁶ is NO₂. In yet another example, R⁷ is H. In one example, X₁ is an oxygen atom, R¹ is Cl, R² is OH, R³ is H, R⁴ is Cl, R⁵ is H, R⁶ is NO₂, R⁷ is H and R⁸ is H.

In one example, D, E and F are each independently CH; X₁ is an oxygen atom, R¹ is any one of Cl, F, Br, I, H, OR¹², SR¹³, NR¹⁴, or R¹⁵, R² is OH, R³ is H, R⁴ is Cl, R⁵ is H, R⁶ is NO₂, R⁷ is H and R⁸ is H. In another one example, D, E and F are each independently CH; X₁ is an oxygen atom, R¹ is Cl, R² is OH, R³ is H, R⁴ is any one of Cl, F, Br, I, H, OR¹², SR¹³, NR¹⁴, or R¹⁵, R⁵ is H, R⁶ is NO₂, R⁷ is H and R⁸ is H. In yet another example, D, E and F are each independently CH; X₁ is an oxygen atom, R¹ is any one of Cl, Br, F, I, H, OR¹², SR¹³, NR¹⁴, or R¹⁵, R² is OH, R³ is H, R⁴ is any one of Cl, Br, F, I, H, OR¹², SR¹³, NR¹⁴, or R¹⁵, R⁵ is H, R⁶ is NO₂, R⁷ is H and R⁸ is H. In one example, D, E and F are each independently CH; X₁ is an oxygen atom, R¹ is Cl, R² is H, OH or OR¹², R³ is H, R⁴ is Cl, R⁵ is H, R⁶ is NO₂, R⁷ is H and R⁸ is H. In yet another example, D, E and F are each independently CH; X₁ is an oxygen atom, R¹ is any one of Cl, Br, F, I, H, OR¹², SR¹³, NR¹⁴, or R¹⁵, R² is H, OH or OR¹², R³ is H, R⁴ is any one of Cl, Br, F, I, H, OR¹², SR¹³, NR¹⁴, or R¹⁵, R⁵ is H, R⁶ is NO₂, R⁷ is H and R⁸ is H. In a further example, D, E and F are each independently CH; X₁ is an oxygen atom, R¹ is Cl, R² is H, OH or OR¹², R³ is H, R⁴ is any one of Cl, Br, F, I, H, OR¹², SR¹³, NR¹⁴, or R¹⁵, R⁵ is H, R⁶ is NO₂, R⁷ is H and R⁸ is H. In yet another example, D, E and F are each independently CH; X₁ is an oxygen atom, R¹ is any one of Cl, Br, F, I, H, OR¹², SR¹³, NR¹⁴, or R¹⁵, R² is H, OH or OR¹², R³ is H, R⁴ is Cl, R⁵ is H, R⁶ is NO₂, R⁷ is H and R⁸ is H. In another example, the compound is niclosamide. In another example, the compound is according to the following structure

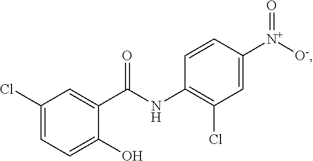

or derivatives thereof.

In another example, the compound claimed herein is used in anti-cancer therapy. In another example, disclosed herein is a kit comprising the compound as disclosed herein, or derivatives thereof, and a further therapeutic agent as described herein.

In an example of the present invention, the compounds outlined above are present as pharmaceutically acceptable salts. The term “pharmaceutically acceptable salt” refers to a salt of the compound of the present invention. Suitable pharmaceutically acceptable salts of the compound of the present invention include acid addition salts which may, for example, be formed by mixing a solution of the compounds of the present invention with a solution of a pharmaceutically acceptable acid such as hydrochloric acid, sulphuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counter anions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydro iodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like. Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide a compound as described herein. A prodrug is a pharmacologically active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. Examples of a masked acidic anion include a variety of esters, such as alkyl (for example, methyl, ethyl), cycloalkyl (for example, cyclohexyl), aralkyl (for example, benzyl, p-methoxybenzyl), and alkylcarbonyloxy alkyl (for example, pivaloyloxymethyl). Amines have been masked as arylcarbonyloxymethyl substituted derivatives which are cleaved by esterases in vivo releasing the free drug and formaldehyde.

Also, drugs containing an acidic NH group, such as imidazole, imide, indole and the like, have been masked with N-acyloxymethyl groups. Hydroxy groups have been masked as esters and ethers. In addition, the compounds having one stereochemistry (e.g., (R)) can often be utilized to produce those having opposite stereochemistry (i.e., (S)) using well-known methods, for example, by inversion.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centres) or double bonds. The racemates, enantiomers, diastereomers, geometric isomers and individual isomers are all intended to be encompassed within the scope of the present invention. Accordingly, the compounds of this invention include mixtures of stereoisomers, especially mixtures of enantiomers, as well as purified stereoisomers, especially purified enantiomers, or stereoisomerically enriched mixtures, especially enantiomerically enriched mixtures. Also included within the scope of the invention are the individual isomers of the compounds as disclosed herein, as well as any wholly or partially equilibrated mixtures thereof. The present invention also covers the individual isomers of the compounds represented by the formulas disclosed herein as mixtures with isomers thereof in which one or more chiral centres are inverted. Also, it is understood that all tautomers and mixtures of tautomers of the compounds as disclosed herein are included within the scope of the compounds disclosed herein.

Racemates obtained can be resolved into the isomers mechanically or chemically by methods known per se. Diastereomers are preferably formed from the racemic mixture by reaction with an optically active resolving agent.

Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as camphorsulfonic acid. Also advantageous is enantiomer resolution with the aid of a column filled with an optically active resolving agent (for example dinitrobenzoylphenylglycine); an example of a suitable eluent is a hexane/ isopropanol/acetonitrile mixture.

The diastereomer resolution can also be carried out by standard purification processes, such as, for example, chromatography or fractional crystallization.

The compounds within the compositions or compounds usable according to the present invention can be present as salts or esters, for example, pharmaceutically acceptable salts or esters. Pharmaceutically acceptable salts of the compounds of the invention include suitable acid addition or base salts thereof. Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkane carboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p- toluene sulfonic acid. Suitable hydroxides include inorganic hydroxides, such as sodium hydroxide, potassium hydroxide, calcium hydroxide, aluminium hydroxide. Alcohols include alkane alcohols of 1 to 12 carbon atoms which may be unsubstituted or substituted, e.g. by a halogen).

The compounds of the present invention may also contain unnatural proportions of atomic isotopes at one or more of the atoms that constitute such compounds. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., isotopes are used due to their ease of preparation and detectability. Further, substitution with isotopes such as deuterium, i.e., ₃H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

All isotopic variations of the compounds and compositions of the present invention, whether radioactive or not, are intended to be encompassed within the scope of the present invention.

The present invention also includes solvate forms of the compounds within the compositions or compounds according to the compounds disclosed herein usable according to the present invention. The terms used in the claims encompass these forms.

The invention furthermore relates to compounds within the compositions of the present invention or compounds according to the compounds disclosed herein usable according to the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

A “pharmaceutical composition” as referred to in the present application comprises at least one compound of the present invention and at least one pharmaceutically acceptable carrier. For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavouring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets can contain from 5% to 80% or from 20% to 70% of the active compound or active compounds. Suitable carriers are magnesium carbonate, magnesium stearate, talc, sugar, lactose, pectin, dextrin, starch, gelatine, tragacanth, methylcellulose, sodium carboxymethyl cellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized moulds, allowed to cool, and thereby to solidify. Liquid form preparations include solutions, suspensions, and emulsions, for example, water or water/propylene glycol solutions. Liquid forms are particularly preferred for topical applications to the eye. For parenteral injection, liquid preparations can be formulated in solution in aqueous polyethylene glycol solution.

Aqueous solutions suitable for oral use can be prepared by dissolving the active component in water and adding suitable colorants, flavours, stabilizers, and thickening agents as desired. Aqueous suspensions suitable for oral use can be made by dispersing the finely divided active component in water with viscous material, such as natural or synthetic gums, resins, methylcellulose, sodium carboxymethyl cellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

The pharmaceutical preparation can be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In one example, according to the method disclosed herein, the compound is to be administered at a concentration (or at a dose) of between 0.01 mg/kg and 5000 mg/kg, between 0.01 mg/kg and 0.5 mg/kg, between 0.05 mg/kg and 10 mg/kg, between 0.1 mg/kg and 100 mg/kg, between 0.1 mg/kg and 1000 mg/kg, between 0.1 mg/kg and 1500 mg/kg, between 0.1 mg/kg and 5 mg/kg, between 1 mg/kg to 2.5 mg/kg, between 2.5 mg/kg to 5 mg/kg, between 5 mg/kg and 10 mg/kg, between 10 mg/kg and 15 mg/kg, between 15 mg/kg and 20 mg/kg, between 17.5 mg/kg and 20 mg/kg, between 5 mg/kg and 7.5 mg/kg, between 7.5 mg/kg and 10 mg/kg, at least about 1mg/kg, at least about 1.5 mg/kg, at least about 1.8 mg/kg, at least about 2 mg/kg, at least about 2.5 mg/kg, at least about 2.8 mg/kg, at least about 3mg/kg, at least about 3.2 mg/kg, at least about 3.5 mg/kg, at least about 4 mg/kg, at least about 4.5mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at about least 9 mg/kg, at least about 9.5 mg/kg, at least about 10 mg/kg, at least about 12.5 mg/kg, at least about 15 mg/kg, at least about 17.5 mg/kg, at least about 19 mg/kg, at least about 20 mg/kg, at least about 50 mg/kg, at least about 100 mg/kg, at least about 150 mg/kg, at least about 200 mg/kg, at least about 250 mg/kg, at least about 300 mg/kg, at least about 350 mg/kg, at least about 400 mg/kg, at least about 450 mg/kg, at least about 500 mg/kg, at least about 550 mg/kg, at least about 600 mg/kg, at least about 650 mg/kg, at least about 700 mg/kg, at least about 750 mg/kg, at least about 800 mg/kg, at least about 850 mg/kg, at least about 900 mg/kg, at least about 950 mg/kg, at least about 1000 mg/kg, at least about 1100 mg/kg, at least about 1200 mg/kg, at least about 1300 mg/kg, at least about 1400 mg/kg, at least about 1500 mg/kg, at least about 1600 mg/kg, at least about 1700 mg/kg, at least about 1800 mg/kg, at least about 1900 mg/kg, at least about 2000 mg/kg, at least about 2100 mg/kg, at least about 2200 mg/kg, at least about 2300 mg/kg, at least about 2400 mg/kg, at least about 2500 mg/kg, at least about 2600 mg/kg, at least about 2700 mg/kg, at least about 2800 mg/kg, at least about 2900 mg/kg, or at least about 300 mg/kg. In one example, the compound is to be administered at a concentration of between about 0.1 mg/kg and about 20 mg/kg. In another example, the compound is 2′,5-dichloro-4′-nitrosalicylanilide (niclosamide) or derivative thereof.

The dosage, frequency and mode of administration of each component of a combination can be controlled independently. For example, one compound, for example niclosamide, is to be administered orally once per day, while the second compound, for example an anti-proliferative or a therapeutic agent, is to be administered intravenously twice per day. Therapy may be given in on-and-off cycles that include rest periods; so that the patient's body has a chance to recovery from any as yet unforeseen side-effects. The compounds may also be formulated together such that one administration delivers both compounds. Accordingly, a compound having any of formulas disclosed herein can be administered simultaneously or within 14 days of administration of, for example, one or more therapeutic agents disclosed herein for the treatment of, for example, any of the diseases listed herein, for example cancer.

Specific methods of the invention further comprise the administration of an additional therapeutic agent (i.e., a therapeutic agent other than a compound of the invention). In one example, the compound as disclosed herein can additionally comprise at least one further therapeutic agent. In another example, 2′,5-dichloro-4′-nitrosalicylanilide (niclosamide), or derivative thereof, is to be administered with a further therapeutic agent. Examples of types of therapeutic agents are, but are not limited to, chemotherapeutic agent, anti-cancer drug capable of targeting cell growth, cytostatic or cytotoxic compounds, survival, angiogenesis, adhesion, migration, invasion, metastasis, cell cycle progression and/or cell differentiation; small molecule drug capable of targeting cell growth, survival, angiogenesis, adhesion, migration, invasion, metastasis, cell cycle progression and/or cell differentiation; bisphosphonate drug for metastatic bone cancer treatment; alkylating substances, anti-metabolites, antibiotics, epothilones, nuclear receptor agonists and antagonists, anti-androgenes, anti-estrogens, platinum compounds, hormones and anti-hormones, interferons and inhibitors of cell cycle-dependent protein kinases (CDKs), inhibitors of cyclooxygenases and/or lipoxygenases, biogeneic fatty acids and fatty acid derivatives, including prostanoids and leukotrienes, inhibitors of protein kinases, inhibitors of protein phosphatases, inhibitors of lipid kinases, platinum coordination complexes, ethyleneimenes, methylmelamines, trazines, vinca alkaloids, pyrimidine analogues, purine analogues, alkylsulfonates, folic acid analogues, anthracendiones, substituted urea, methylhydrazin, derivative peptide therapeutic agents and combinations thereof.

Examples of therapeutic agents are, but are not limited to, are, but are not limited to, 10-hydroxycamptothecin, abraxane, acediasulfone, aclarubicine, aklavine hydrochloride, ambazone, amsacrine, aminoglutethimide, anastrozole, ancitabine hydrochloride, L-asparaginase, azathioprine, bleomycin, bortezomib, busulfan, calcium folinate, carbop latin, carpecitabine, carmustine, celecoxib, chlorambucil, cisplatin, cladribine, colchicine, cyclophosphamide, cytarabine, dacarbazine, dactinomycin dapsone, daunorubicin, dibrompropamidine, diethylstilbestrole, docetaxel, doxorubicin, emetine, enediynes, epirubicin, epothilone B, epothilone D, estramucin phosphate, estrogen, ethinylestradiole, etoposide, epirubicin hydrochloride, faslodex, flavopiridol, floxuridine, fludarabine, fluorouracil, 5-fluorouracil, fluoxymesterone, flutamide fosfestrol, furazolidone, gambogic acid amide, gambogic acid, gemcitabine, gonadotropin releasing hormone analog, herceptin, hexamethylmelamine, hydroxycarbamide, hydroxymethylnitrofurantoin, hydroxyprogesteronecaproat, hydroxyurea, idarubicin, idoxuridine, ifosfamide, interferon γ, irinotecan, imatinib, irinotecan, letrozole, leuprolide, lomustine, lurtotecan, mafenide sulfate olamide, mechlorethamine, medroxyprogesterone acetate, megastrolacetate, melphalan, mepacrine, mercaptopurine, methotrexate, metronidazole, mitomycin C, mitoxanthrone hydrochloride, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, oleomucin, oxolinic acid, oxaliplatin, ouabain, pentamidine, pentostatin, phenazopyridine, phthalylsulfathiazole, phenylmercuric acetate, picropodophyllotoxin, pipobroman, prednimustine, prednisone, preussin, pristimerin, procarbazine, pyrimethamine, quinacrine hydrochloride, raltitrexed, rapamycin, rotenone, rofecoxib, rosiglitazone, raloxifen, salazosulfapyridine, scriflavinium chloride, semustine streptozocine, sulfac arb amide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, temozolimide, teniposide, teniposide, testolactone, testosteronpropionate, thimerosal, thioguanine, thiotepa, imidazole, topotecan, trastuzumab, triaziquone, treosulfan, trimethoprim, trofosfamide, UCN-01, vinblastine, vinblastine sulfate, vincristine, vincristine sulfate, vindesine, vinorelbine, and zorubicin, or their respective derivatives or analogues thereof. In one example, the chemotherapeutic agent is, but is not limited to, cisplatin, etoposide, abraxane, trastuzumab, gemcitabine, imatinib, irinotecan, oxaliplatin, bortezomib, methotrexate, chlorambucil, doxorubicin, dacarbazine, cyclophosphamide, paclitaxel, 5-fluorouracil, gemcitabine, vincristine, docetaxel, vinorelbine or epothilone B.

The present disclosure also describes combination therapies and compositions, that is to say that the compound, as described herein, may be administered simultaneously, sequentially or separately, or combinations thereof, from a further therapeutic agent. Thus, in one example, the administration is simultaneous, that is to say that both the compound disclosed herein, for example niclosamide, and the further therapeutic agent are to be administered at the same time. In another example, the compound disclosed herein, for example niclosamide, and the further therapeutic agent are to be administered separately. In yet another example, the recombinant dermatopontin and the further therapeutic agent are to be administered sequentially, that is to say that, for example niclosamide, can be administered first, followed by administration of the further therapeutic compound. Alternatively, in another example, the further therapeutic agent can be administered first, followed by the administration of, for example, niclosamide. In yet another example, a first therapeutic agent is to be administered, after which the compound as disclosed herein, for example niclosamide, is administered, followed by the administration of a second therapeutic agent. In a further example, one therapeutic agent is to be administered, followed by the subsequent administration of the recombinant dermatopontin simultaneously with a second therapeutic agent. In one example, the further therapeutic agent is to be administered simultaneously, sequentially or subsequently after administration of a compound as defined herein. In another example, the further therapeutic agent is to be administered simultaneously, sequentially or subsequently after administration of 2′,5-dichloro-4′-nitrosalicylanilide (niclosamide).

A compound according to the invention can be administered by various well known routes, including oral, rectal, intragastrical, intracranial and parenteral administration, e.g. intravenous, intramuscular, intranasal, intradermal, subcutaneous, depot injections and similar administration routes. Depending on the route of administration different pharmaceutical formulations are required and some of those may require that protective coatings are applied to the drug formulation to prevent degradation of a compound of the invention in, for example, the digestive tract. In one example, a method of treatment can comprise one or more administration routes, which can be, but are not limited to, orally, intraadiposally, intraarterially, intraarticularly, intracranially, intradermally, intralesionally, intramuscularly, intranasally, intraocularally, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in crmes, in lipid compositions, via a catheter, via a lavage, via continuous infusion, via infusion, via inhalation, via injection, via local delivery, via localized perfusion, bathing target cells directly, or any combination thereof. In one example, the compound as disclosed herein is administered orally. In another example, the compound disclosed herein is administered percutaneously or topically.

According to the administration routes as disclosed above, a compound of the invention can be formulated as a syrup, an infusion or injection solution, a tablet, a capsule, a capslet, lozenge, a liposome, a suppository, a plaster, a band aid, a retard capsule, a powder, or a slow release formulation. In one example, the compound 2′,5-dichloro-4′-nitrosalicylanilide (niclosamide) or derivative thereof, is formulated according to, but not limited to, any one of the following: tablet, caplet, capsule, hard capsule, soft capsule, soft elastic gelatine capsule, hard gelatine capsule, cachet, troche, lozenge, dispersion, suppository, ointment, cataplasm, poultice, paste, powder, dressing, cream, plaster, solution, patch, aerosol, nasal spray, inhaler, gel, suspension, aqueous liquid suspension, non-aqueous liquid suspension, oil-in-water emulsion, water-in-oil liquid emulsion, solution, sterile solid, crystalline solid, amorphous solid, solid for reconstitution or combinations thereof.

The dosage of the claimed compounds depends on several factors, including, but no limited to the administration method, the disease to be treated, the severity of the disease, whether the disease is to be treated or prevented, and the age, weight, and health of the patient to be treated.

As described above, the compound described herein may be administered in various forms, for example, but not limited to, orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. In the following paragraphs, for exemplary and illustrative purposes, the dosages for niclosamide are described. A person skilled in the art will appreciate that if an alternative compound is substituted for niclosamide, the correct dosage can be determined by examining the efficacy of the compound in cell proliferation assays, as well as its toxicity in mammals, for example humans. For topical administration, niclosamide is provided in a 1-250 g/L solution, cream, or gel. For parenteral or enteral administration, niclosamide is dosed at about 0.1-50 mg/kg/day. For oral administration, niclosamide is dosed at about 10-3000 mg/day.

For oral administration, the dosage of a compound disclosed herein, for example niclosamide, is about 0.001 mg to 3000 mg per dose administered. In one example, the oral dosage is about 0.05 mg to 2000 mg. In another example, the oral dosage is about 0.5 mg to 1000 mg. In another example, the oral dosage is to be administered one to ten times daily. In another example, the oral dosage is to be administered one to five times daily. In another example, the oral dosage is to be administered one to three times daily. The doses may be given in cycles, such that there are periods during which time the compounds disclosed herein, for example niclosamide, are not administered. In one example, this period could be, for example, any of the following, but not limited to, about a day, a week, a month, or a year or more.

For compositions adapted for rectal use for preventing infection, a somewhat higher amount of a compound is usually preferred. Thus, in one example, the rectal dosage of the compounds as disclosed herein, for example a dosage of a niclosamide, is about 0.5 mg to 2500 mg per dose. In another example, the rectal dosage is about 0.5 mg to 1500 mg. In another example, the rectal dosage is to be administered one to four times daily. Treatment durations are as described above for oral administration.

For intravenous or intramuscular administration of the compounds disclosed herein, for example niclosamide, a dose of for example, about 0.01 mg/kg to about 50 mg/kg body weight per day is recommended. In another example, the recommended dose is about 0.05 mg/kg to about 15 mg/kg. In yet another example, the recommended dose is about 0.1 mg/kg to about 5 mg/kg.

A compound can be administered daily for up to about 6 to 12 months or more. It may be desirable a compound to be administered over a one to three hour period. In one example, this period may be extended to last 24 hours or more.

For inhalation, the compounds disclosed herein, for example niclosamide, are to be administered at a dose of about 0.001 mg to 5000 mg daily. In one example, the inhaled dose about 0.5 mg to 2000 mg daily.

For topical/percutaneous administration of the comoiunds disclosed herein, for example niclosamide, a dose of about 1 mg to about 5 mg is to be administered one to ten times daily for a time span of one week to 12 months.

Pharmaceutical forms for the administration of a compound of the invention are forms suitable for injectable use and include sterile aqueous solutions or dispersions and sterile powders for the extemporaneous preparation of sterile injectable solutions or dispersion. In all cases the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half-life in the circulation, if compared to the free drug and a prolonged more even release of the enclosed drug.

Sterilization of infusion or injection solutions can be accomplished by any number of art recognized techniques including but not limited to addition of preservatives like anti-bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions. Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum-dried or freeze-dried as necessary. Diluents of the present invention are, but are not limited to, water, physiological acceptable buffers, physiological acceptable buffer salt solutions or salt solutions. Carriers are, but are not limited to, cocoa butter and vitebesole. Besides the excipients mentioned already above, also the following excipients can be chosen, without limitation, to be used with the various pharmaceutical forms of a compound of the invention:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscaramellose, sodium methyl cellulose, agar, bentonite, alginic acid, carboxymethyl cellulose, polyvinyl pyrrolidone and the like.

Other suitable excipients can be determined according to knowledge known in the art. It is to be understood that depending on the severity of the disorder and the particular type which is treatable with one of the compounds of the invention, as well as on the respective patient to be treated, e.g. the general health status of the patient, etc., different doses of the respective compound are required to elicit a therapeutic or prophylactic effect. The determination of the appropriate dose lies within the discretion of the attending physician. As is known in the art, the pharmaceutically effective amount of a given composition will also depend on the administration route. In general the required amount will be higher, if the administration is through the gastrointestinal tract; e.g. by suppository, rectal, or by an intragastric probe, and lower if the route of administration is parenteral, e.g. intravenous.

Within the meaning of this invention, a combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week. This invention also envisions the quarterisation of any basic nitrogen- containing groups of the compounds disclosed herein. Water or oil-soluble or dispersible products may be obtained by such quarterisation.

The compounds can be formulated for oral, intravenous, intrathecal or intraparenchymal administration. As the compound as disclosed herein and its derivatives can be administered orally, these compounds can thus be formulated for oral administration. In this case, appropriate amounts of the compound, or derivatives thereof, can easily combined for simultaneous administration so that the most effective concentrations are achieved simultaneously at the site of the disease, for example, the tumour.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by examples and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### Experimental Section

### Niclosamide Sensitises p53 Deficient Cells

It was shown that the compounds as disclosed herein, for example niclosamide or derivatives thereof, induce a preferential sensitization of p53-deficient cells. Niclosamide results in increased apoptosis of HCT116 p53 knockout cells and normal human fibroblasts in which p53 gene is knocked down using p53-specific shRNAs compared to HCT116 and normal human fibroblasts harbouring wildtype p53. The loss in viability of cells without wildtype p53 functions is translated to decreased spheroid growth and reduced tumour growth in nude mice.

Screening of Drug Compounds using p53^{+/+} and p53^{-/-} Cells

Stably expressing H2B-GFP-HCTp53^{+/+} cell (wild type p53 cells) and H2B-RFP-HCTp53^{-/-} cells (cells lacking p53) were grown in co-culture to screen drug compounds sensitizing either wild type p53 cells or p53-negative cells. Briefly, 200 cells of each

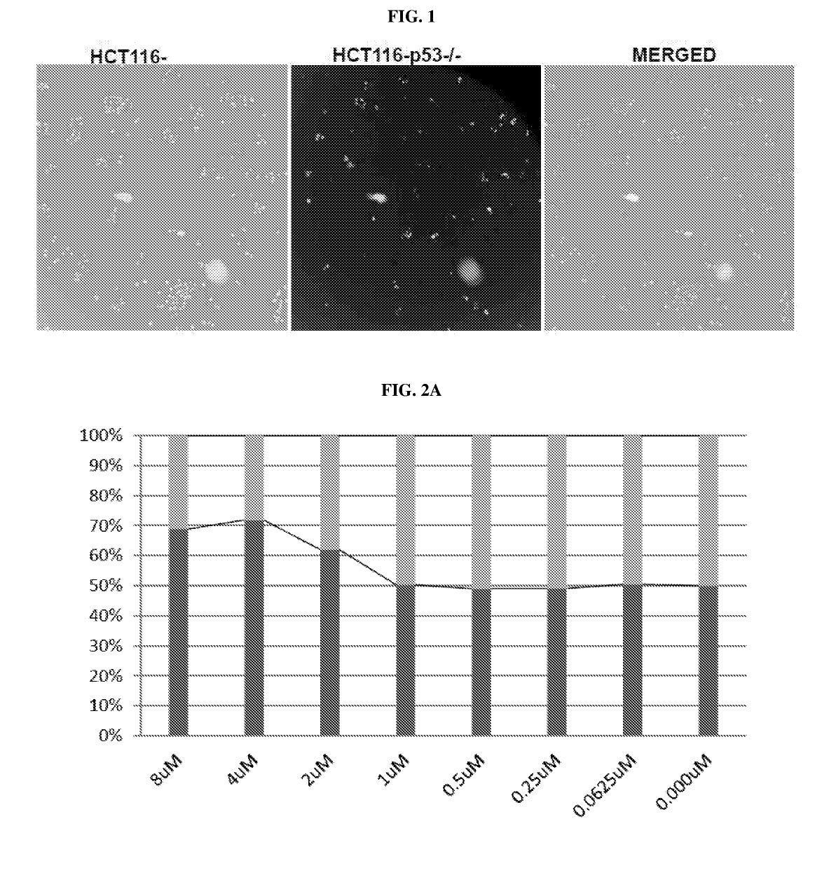

HCT116p53^{+/+} and HCT116p53^{-/-} were plated in a pre-coated 384-well drug plates at final cell density of 400 cells per well at a final volume of 80 μl per well. After plating cells into respective wells, plates were incubated for 6 days. After 6 days of drug incubation, plates were imaged in an Incell Analyzer 200 machine by performing a multi-channel read, that is applying FITC staining to count GFP positive cells and Texas Red staining to count RFP positive cells. After imaging, the raw data was processed through the Incell-Investigator software to count the number of GFP positive and RFP positive cells. The data presented in FIG. 1 is the raw data from large scale imaging.

Chemical biology screen using co-culture of HCT116 p53 identifies niclosamide as selectively targeting HCT116 p53^{-/-} cells. Wildtype HCT116 cells expressing H2B-GFP were co-cultured with HCT115 p53^{-/-} cells expressing H2B-RFP. Cells were treated with niclosamide and incubated for 48 hours before allowing cells to recover for 7 days in drug free media. The surviving cells were imaged using INCELL Analyzer 2000 to determine the relative percentages of GFP (HCT116 wildtype) and RFP (HCT116 p53^{-/-}) cells.

### Secondary Screen to Validate Potential Drug Compounds

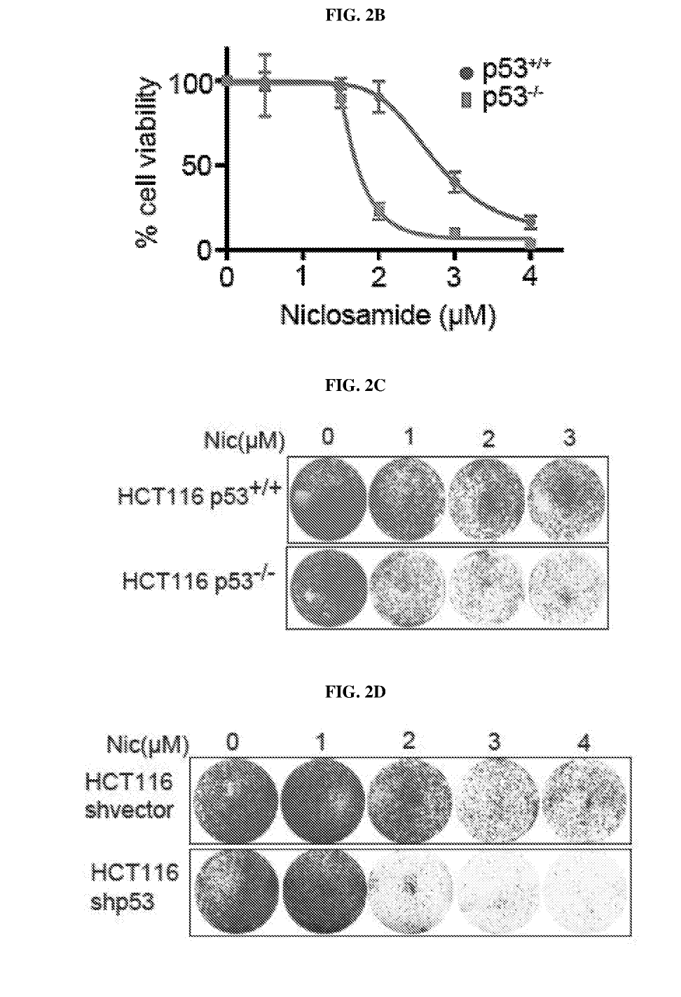

2000 cells each of H2B-GFP-HCT116p53^{+/+} and H2B-RFP-HCT116p53^{-/-} were grown as co-culture in 96-well plates in a total volume of 100 ∥l. The next day, these cells were either DMSO treated or treated with 7 different concentrations of niclosamide (as shown in FIG. 2b). After 48 hours of drug incubation, the cell media was changed to start recovery. After 6 days of recovery, the cells were imaged in an Incell Analyzer 2000 machine and the percentage of viable cells was determined and plotted against the drug concentrations used in the experiment.

### Niclosamide Sensitivity Analysis in Isogenic Cell Lines

In another experiment, negative control scrambled shRNA (Addgene Plasmid #1864), p53-targeting pLKO-p53-shRNA-941 (Addgene Plasmid #25637) and p53-targeting shp53 pLKO.1 puro (Addgene Plasmid #19119) were used to produce different lentiviruses, each containing one of the shRNA constructs listed previously. Using the HCT116P53^{+/+} cell line, cells were infected either with a control virus containing the negative control scrambled shRNA or with two different viruses targeting the p53 gene. In some instances, the HCT 116 cell line was infected with two independent shRNA, each separately targeting p53 expression. These independent p53 expression targeting shRNA were named shRNA(I) and shRNA (II), accordingly. The one or more viruses were used at a multiplicity of infection of 2 (MOI 2) in a hexadimethrine bromide (polybrene)-mediated viral transduction. After 18 hours of infection, the culture media was changed. After two days of infection, cells were added with cell culture media containing puromycin (1 μg/ml). After two weeks, the newly formed antibiotic-resistant clones were trypsinised and transferred into new cell culture dish with puromycin. These cells were tested for p53 depletion by Western blot analysis. Detection of a p53-knockdown of up to 90% was detected by immunoblotting.

HCT116-p53^{+/+} NTshRNA, HCT116-shp53 (I) and HCT116-sh p53 (II) were further used to analyse cell viability after niclosamide treatment by colony forming assay. Briefly, 10,000 cells were plated in 6-well cell culture plates with 2 ml cell culture media. The next day, cells were treated either with DMSO as a negative control or with one of five different concentrations of the niclosamide, 0.5 μM, 1 μM, 1.5 μM, 2 μM and 3 μM. After 48 hours of drug incubation, the drug containing media was replaced with new cell culture medium and the cells were grown for another 7 days. On day 8th of the recovery, the cells were stained with crystal violet and gluteraldehyde solution for 2 hours and de-stained in plain water.

It is shown that niclosamide treatment results in a decrease in the viability of HCT116 p53^{-/-} cells to a greater extent than the HCT116 p53 wildtype cells after 48 hours of drug treatment, whereas DMSO treated controls show equivalent cell viability of HCT116 wildtype and HCT116 p53^{-/-} cells. This is demonstrated in a cell viability assay (FIG. 2a) and in a colony formation assay (FIG. 2b)

### Niclosamide Sensitises p53 Deficient Human Skin Fibroblast Cells

Human skin fibroblast cells (TOBA cells) were infected either with a control shRNA lentivirus or a p53-targeting shRNA virus (as described in the preceding section). After generation of a stable p53-knockdown (using puro selection), cells were analysed for cell viability in response to different doses of niclosamide. Briefly, wild type Toba cells, and p53 depleted cells were plated at 20,000 cell density in 6 well plates. Next day, cells were either DMSO treated or treated with varying doses of niclosamide. After 48 hours of drug incubation, the drug containing media was replaced with new cell culture medium to start recovery. After 8 days of recovery, the cells were imaged in light microscope at 10× objective lens.

As described above, DMSO and niclosamide-treated cells were analysed for the presence of apoptotic cell death by Annexin V staining. All cells in a well, including both rounded floating (that is, dead or dying cells) and adherent cells (that is, live cells) were collected carefully by trypsinisation and centrifuged at 400 g for 5 minutes. The pellet was resuspended in 200 μl of incubation buffer and after re-suspension, centrifuged at same speed as above to collect the washed sample pellets. These pellets were then incubated with Annexin V solution premixed with the incubation buffer (10 μl of Annexin V mixed in 990 μl of incubation buffer) for 20 minutes in dark at room temperature. After incubation, 250 μl of incubation buffer was added to each pellet and FACS analysis performed to measure the amount of dead cells.

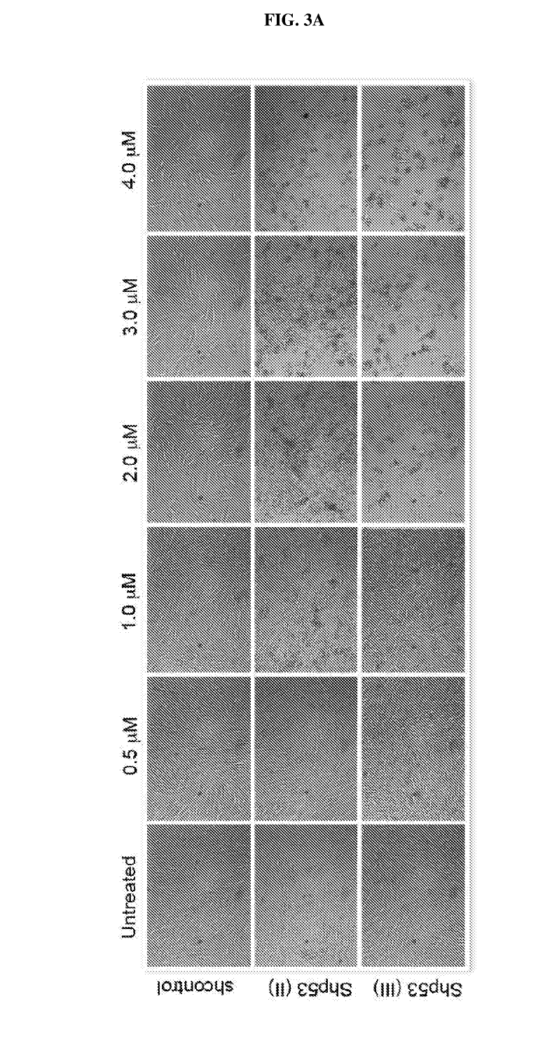

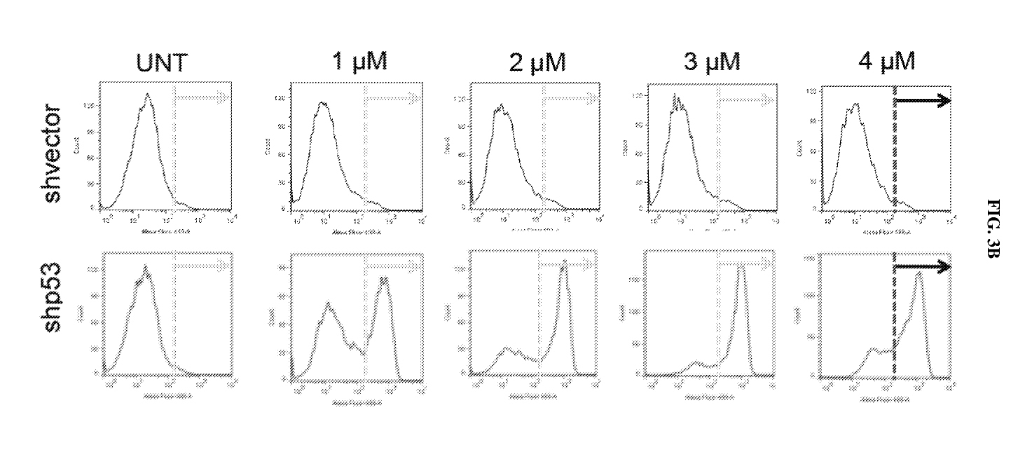

It was shown that niclosamide results in a greater extent of apoptosis in p53-deficient human fibroblasts (FIG. 3) compared to normal wildtype human fibroblasts, suggesting that the increased sensitivity of niclosamide on p53-deficient cells is consistent across different genetic background.

### Niclosamide Inhibits Spheroid Growth in p53 Deficient Colorectal Carcinoma Cells

H2B-GFP- HCT116p53^{+/+} and H2B-RFP-p53^{-/-} cells were grown as co-culture in a ultralow attachment 96-well cell culture plates to promote the spheroid formation. Briefly, to generate 3D multicellular spheroids, H2B-GFP expressing wild type (WT) p53 cells and H2B-RFP expressing p53-negative cells were each seeded at 250 cells per 100 ∥l culture media into 96-well round-bottomed plate and spun down (1000 g, 5 min). The seeded cells were cultured for two days in 5% CO₂ humidified atmosphere at 37° C. On third day, cells were either DMSO treated or treated with different doses of niclosamide. After 48 hours of drug incubation, spheroids were diluted to non-toxic concentrations via media exchange. Treated spheroids were then further cultured for 6 more days. Spheroids were imaged in Incell analyser 2000 with FITC and Texas-Red channels.

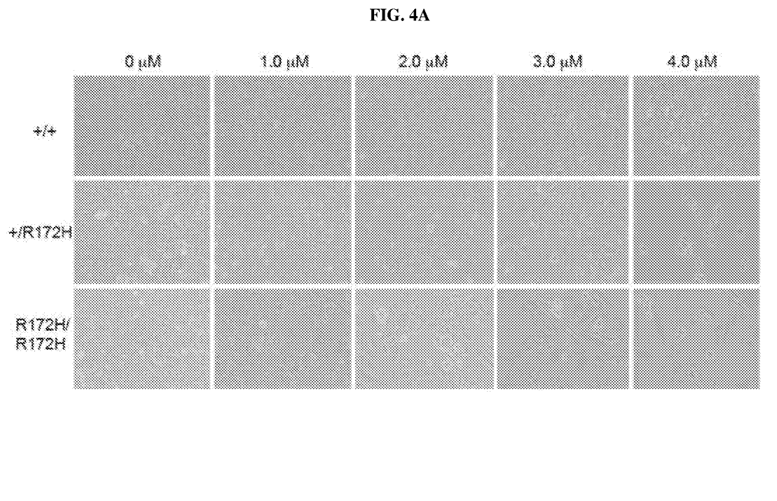

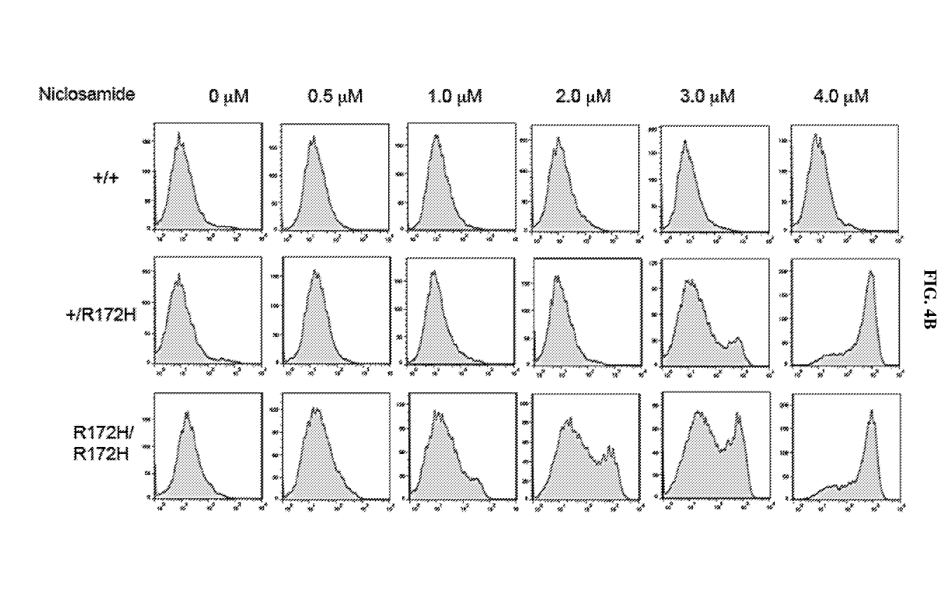

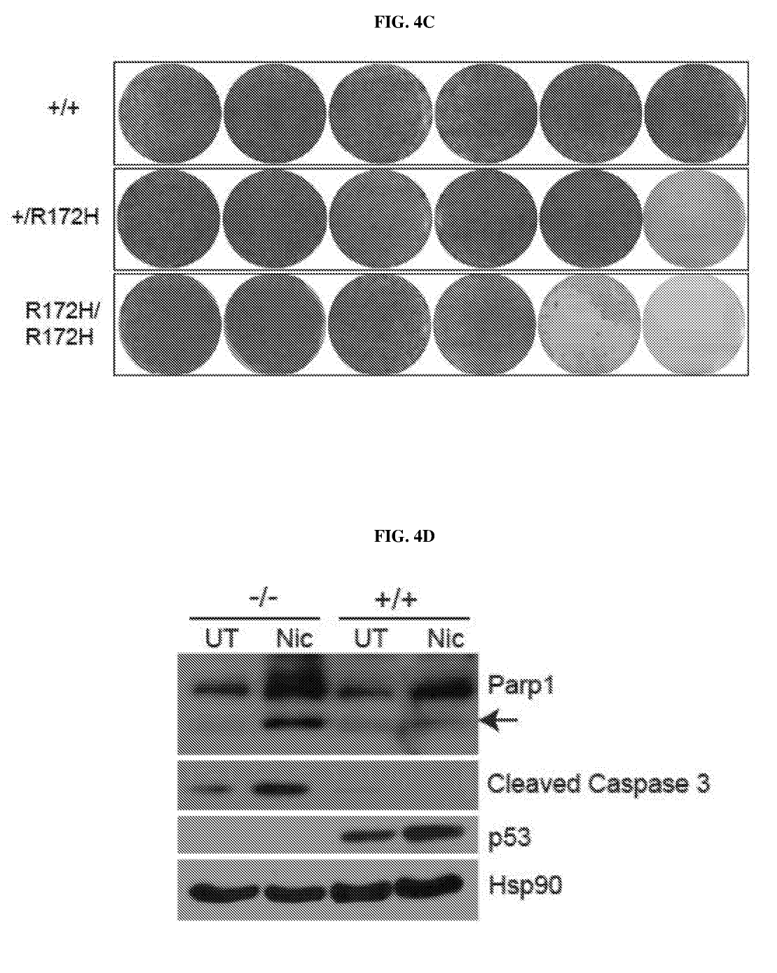

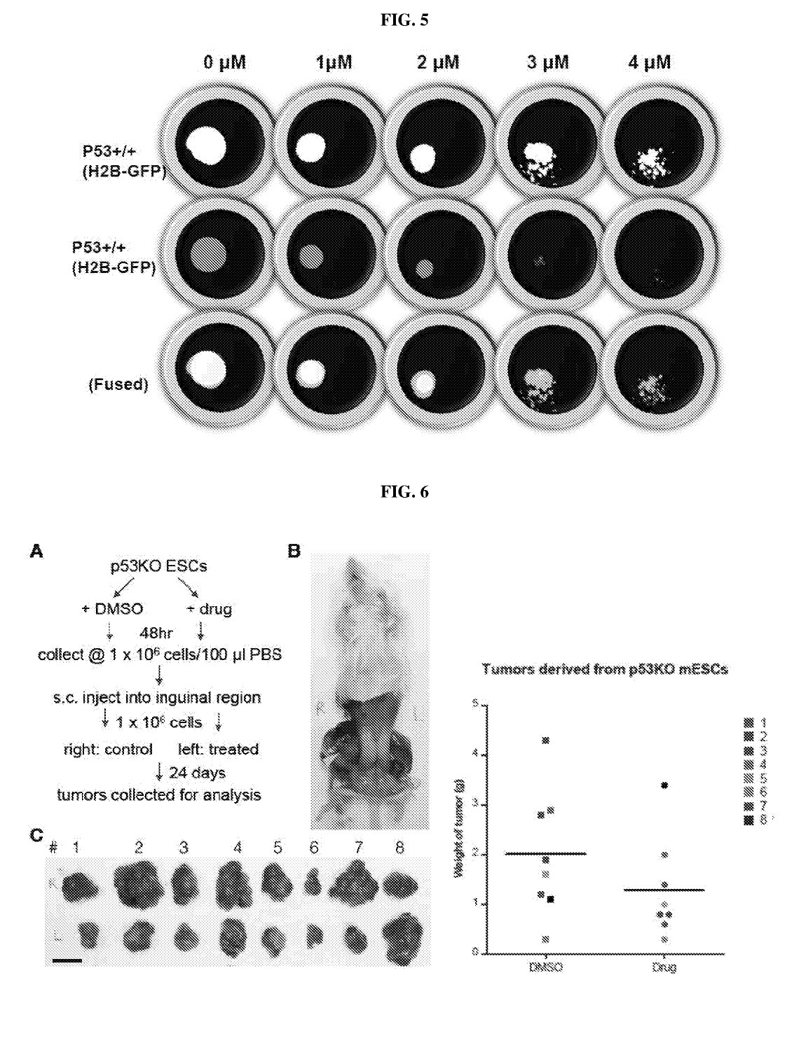

It was shown that 3D culture spheroids assay served as a mimic of in vivo tumour growth and may more accurately reflect the effects of drugs on tumour reduction than 2D culture. It was demonstrated that niclosamide results in a greater reduction of 3D p53-deficient HCT116 spheroid growth compared to wildtype spheroid (FIG. 4) when compared to normal wildtype human spheroids.

### Niclosamide Strongly Induces Apoptotic Cell Death in p53 Null and p53 Mutant Cancer Cells

It was shown in in vitro cell culture that niclosamide had a stronger apoptotic effect on cancer cells having a p53 mutation or p53 null cancer cells, compared to cancer cells with a functional p53 gene. To this end, various cancer cell lines/types were seeded according to their growth rate to be confluent at the same time. These cell lines were each treated with differing niclosamide concentrations for 48 hours. The cells were then allowed to recover in their respective media for 6 days, after which apoptotic cell death assays were performed. The resulting percentage of apoptotic cells in each sample were plotted on a 3D graph (data not shown). It was shown that both p53 null and p53 mutant cancer cells were strongly susceptible to treatment with niclosamide in a dose dependent manner, with certain cell types showing immediate sensitivity to niclosamide treatment. It was also further shown that niclosamide differentially reduced cell viability of the remaining cells in certain cell lines, indicating that cells which survived the initial niclosamide treatment show a delayed therapeutic response to the initial niclosamide treatment (data not shown). It is noted that these less viable cells would expire after treatment, without further doses of niclosamide being required, indicating the presence of a possible secondary killing effect of niclosamide

### Niclosamide Targets p53 Deficient Tumours in Mouse

Mouse embryonic stem cells harbouring a p53K0 mutation were either treated with DMSO or treated with niclosamide at a concentration of 2 μM for 48 hours. The resulting trypsinised cells were then resuspended in Phosphate buffer Saline. The cells were counted and resuspended to result in a final cell concentration of 1×10⁶ cells/100 μl. Immediately after counting, the cells were transferred to animal facility for nude mice injection. 100 μl of DMSO treated cells were injected into the right inguinal region, while 100 μl niclosamide treated cells were injected into the left inguinal region of each mouse. After 24 days of the injection, tumour growth was observed. The resulting teratomas were surgically removed analysed to observe the ability of niclosamide to inhibit tumour growth.

The experiments were done to demonstrate that niclosamide has an anti-tumour effect in vivo. Therefore, an in vivo tumour model in nude mice was utilised. As a control, mouse embryonic stem (mES) cells were injected into nude mice and the mice monitored for the presence of tumour growth (teratomas). mES cells treated with niclosamide or DMSO treated control cells were injected into nude mice and tumour growth monitored after 3 weeks. The results demonstrated that niclosamide is an effective anti-cancer agent, as it results in significant reduction in the size of the teratomas, compared to the size of the teratomas in mice treated with the DMSO control only (data not shown).

Experiments were also performed using a patient-derived xenograft mouse model. In these experiments, ovarian tumour samples derived from patient biopsy samples were implanted into either the left or right flank of the mouse. These tumours were allowed to establish in the mouse and the tumour volume was measured according to standard practice every 2 days post implantation. After reaching a minimum threshold size of about 200 mm³, the mice were treated once, intravenously, with niclosamide at a concentration of 150 mg/kg. The change in tumour volume was measured for further 30 days post treatment. As shown in the previous in vivo experiment of a similar set up, it was shown that the treatment of the tumour-bearing mice with niclosamide resulted in a reduction in tumour growth, compared to the negative control mice which had been treated with PBS.

### Cell Culture and Spheroid Culture Media Used

Toba wild type cells and shp53 depleted Toba cell lines

Cell culture medias used: DMEM High glucose medium supplemented with 10% Fetal bovine serum and Penstrep antibiotics; Mycos 5A supplemented with 10% fetal bovine serum and Penstrep antibiotic.

Cell lines utilised are HCT116p53^{+/+}, HCT116p53^{-/-}, HCT116p53^{+/+} H2B-GFP, HCT116p53^{-/-}H2B-RFP. HCT116 p53^{-/-} were generated by the Bert Vogelstein lab (Bunz, F., Dutriaux, A., Lengauer, C., Waldman, T., Zhou, S., Brown, J. P., Sedivy, J. M., Kinzler, K. W., and Vogelstein, B. (1998) Science 282, 1497-1501). The HCT116 p53^{-/-} cell line is freely available for academic research and widely circulated. The H2B-GFP and H2B-RFP cell lines in/based on the HCT116 background were generated in house.

Puromycin was used in respective cell culture media after 2 days of lentiviral transduction to select p53 depleted TOBA cell line and HCT116 cell lines.

1. A method of treating cancer in a patient comprising administering a pharmaceutically effective amount of a compound as defined in formula I: 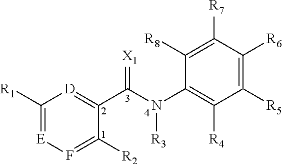
or derivative thereof, wherein
D is N or CR⁹; E is N or CR¹⁰; F is N or CR¹¹;
R¹ is H, halide, OR¹², SR¹³, NR14, R¹⁵, 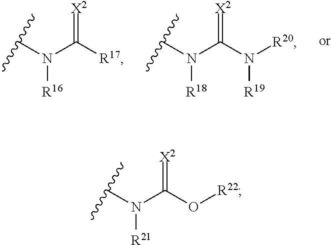
R² is H, OH or OR¹²;
R³ is H, C₁₋₇ alkyl, C₂₋₇ alkenyl, C₂₋₇ alkynyl, C₂₋₆ heterocyclyl; C₆₋₁₂ aryl, C₇₋₁₄ alkaryl; C₃₋₁₀ alkheterocyclyl, or C₁₋₇ heteroalkyl; or R² and R³ combine to form a six-membered ring in which position 1 is connected to position 4 by 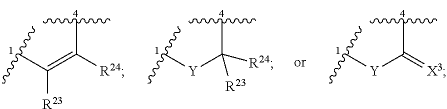
R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, 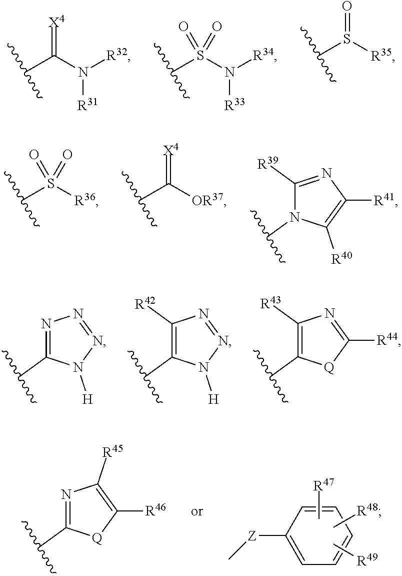
each X¹, X², X³, and X⁴ is, independently, O, S; or NR³⁸;
Y is CR²⁵R²⁶, O, S, or NR²⁷;
Z is O, S, or CR⁵⁰R⁵¹;
each Q is, independently, O, S, or NR⁵² ;
R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂;
R¹² and R¹³ are each, independently, acyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵⁰, are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, wherein the cancer comprises p53-deficient cells or cells wherein p53 is non-functional.

2. The method of claim 1, wherein X¹ is an oxygen atom, R² is OH and R³ is H.

3. The method of claim 1, wherein R¹ is H or halide; optionally wherein R¹ is Cl.

4. (canceled)

5. The method of claim 1, wherein R⁴ and R⁸ are independently H, halide or CF₃; optionally wherein R⁴ is Cl or wherein R⁸ is H.

6. (canceled)

7. (canceled)

8. The method of claim 1, wherein R⁵, R⁶ and R⁷ are independently H, halide, NO₂, CO₂H, SO₃H, CF₃ or CN.

9. The method of claim 8, wherein R⁵ is H; optionally wherein R⁶ is NO₂ or wherein R⁷ is H.

10. (canceled)

11. (canceled)

12. The method of claim 1, wherein X¹ is O, R¹ is Cl, R² is OH, R³ is H, R⁴ is Cl, R⁵ is H, R⁶ is NO₂, R⁷ is H and R⁸ is H.

13. The method of claim 1, wherein the compound is 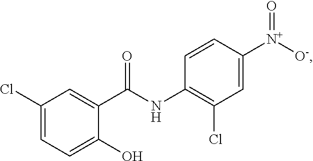
or derivatives thereof.

14. (canceled)

15. (canceled)

16. (canceled)

17. (canceled)

18. The method of claim 1, wherein the cancer is selected from a group consisting of leukemia, lymphoma, myeloma, skin cancer such as basal cell carcinoma (BCC), squamous cell carcinoma (SCC) or melanoma, breast cancer, head and neck cancer, such as brain cancer, colorectal cancer, colon cancer, rectal cancer, lung cancer, such as non-small cell lung cancer (NSCLC), ovarian cancer, renal cancer, prostate cancer, liver cancer, and human papilloma virus (HPV)-associated cancer such as cervical cancer; or wherein the cancer is selected from a group consisting of leukaemia, lymphoma, myeloma, skin cancer such as basal cell carcinoma (BCC), squamous cell carcinoma (SCC) or melanoma, head and neck cancer, such as brain cancer, colorectal cancer, colon cancer, rectal cancer, lung cancer, such as non-small cell lung cancer (NSCLC), ovarian cancer, renal cancer, prostate cancer, liver cancer, and human papilloma virus (HPV)-associated cancer such as cervical cancer.

19. (canceled)

20. The method of claim 1, further comprising administering a further therapeutic agent.

21. The method of claim 20, wherein the further therapeutic agent is selected from a group consisting of chemotherapeutic agent, anti-cancer drug capable of targeting cell growth, survival, angiogenesis, adhesion, migration, invasion, metastasis, cell cycle progression and/or cell differentiation; small molecule drug capable of targeting cell growth, survival, angiogenesis, adhesion, migration, invasion, metastasis, cell cycle progression and/or cell differentiation; bisphosphonate drug for metastatic bone cancer treatment; peptide therapeutic agent and combinations thereof.

22. The method of claim 21, wherein the chemotherapeutic agent is selected from a group consisting of doxorubicin, cisplatin, etoposide, abraxane, trastuzumab, gemcitabine, imatinib, irinotecan, oxaliplatin, bortezomib, methotrexate, chlorambucil, doxorubicin, dacarbazine, cyclophosphamide, paclitaxel, 5-fluorouracil, gemcitabine, vincristine, docetaxel, vinorelbine and epothilone B.

23. The method of claims 20, wherein the further therapeutic agent is to be administered simultaneously, sequentially or subsequently after administration of the compound.

24. (canceled)

25. (canceled)

26. A method of screening to identify a compound that selectively targets a first cell type or a second cell type, wherein the first cell type is a p53-deficient cell or a cell wherein p53 is non-functional, and wherein the second cell type is a p53-positive cell, and wherein the first cell type is labelled with a first detectable marker and wherein the second cell type is labelled with a second detectable marker, the method comprising:
A. contacting the first and second cell types with the compound; and
B. determining the relative amounts of the first and second detectable markers;
wherein the first and second detectable marker are independently detectable and wherein a relative increase in the amount of the first marker in comparison to the amount of the second marker is indicative of a compound selectively targeting a p53-positive cell and wherein a relative increase in the amount of the second marker is indicative of a compound selectively targeting a p53-deficient cell or a cell wherein p53 is non-functional.

27. The method of claim 26, wherein the detectable marker is selected from the group consisting of a fluorescent compound, a radioisotope compound, a non-radioisotope compound, a bioluminescent compound, a chemiluminescent compound, a metal chelator compound, a chromogenic compound, an X-radiographic compound, and an enzyme.

28. The method of claim 27, wherein said fluorescent compound is selected from the group consisting of a Green Fluorescent Protein (GFP), Red Fluorescent Protein (RFP) fluorescein, rhodamine, phycoerytherin, phycocyanin, allophycocyanin, o-phthaldehyde and fluorescamine.

29. The method according to claim 28, wherein said first cell type is labelled with Red Fluorescent Protein (RFP) and said second cell type is labelled with Green Fluorescent Protein (GFP).

30. The method according to claim 26, wherein said cell types are contacted with the compound in vitro or ex vivo according to a method of treating cancer in a patient comprising administering a pharmaceutically effective amount of a compound as defined in formula I: 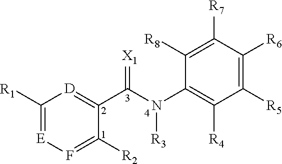
or derivative thereof, wherein
D is N or CR⁹: E is N or C¹⁰; F is N or CR¹¹;
R¹ is H, halide, OR¹², SR¹³, NR¹⁴, R¹⁵, 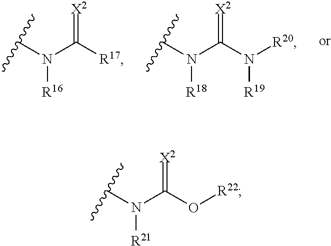
R² is H, OH or OR¹²;
R³ is H, C₁₋₇ alkyl, C₂₋₇ alkenyl, C₂₋₇ alkynyl, C₂₋₆ heterocycyl; C₆₋₁₂ aryl, C₇₋₁₄ alkaryl; C₃₋₁₀ alkheterocyclyl, or C₁₋₇ heteroalkyl; or R² and R³ combine to form a six-membered ring in which position 1 is connected to position 4 by 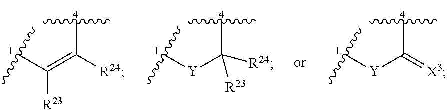
R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl:
R⁵, R⁶, and R⁷ are each, independently selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, 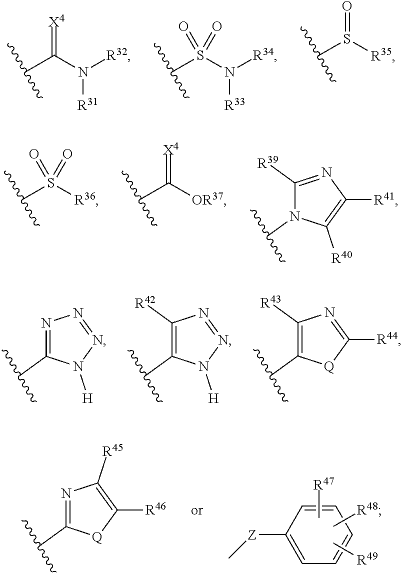
each X¹, X², X³, and X⁴ is, independently, O, S; or NR³⁸;
Y is CR²⁵R²⁶, O, S, or NR²⁷;
Z is O, S, or CR⁵⁰R⁵¹;
each Q is, independently, O, S, or NR⁵² ;
R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂;
R¹² and R¹³ are each, independently, acyl, C₁₋₇ alkyl, C₂₋₇ alkenyl, C₂₋₇ alkynyl, C₂₋₆ heterocyclyl. C₆₋₁₂ aryl, C₇₋₁₄ alkaryl, C₃₋₁₀ alkheterocyclyl, or C₁₋₇ heteroalkyl;
R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl or C₁₋₇ heteroalkyl; R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently H, halide, CN, NO₂, CF₃, C₁₋₁₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, wherein the cancer comprises p53-deficient cells or cells wherein p53 is non-functional.

31. The method of claim 26, wherein said relative amounts of the first and second marker are determined using fluorescence-activated cell sorting (FACS) or quantitative imaging microscopy.

32. A kit comprising the compound and a further therapeutic agent wherein the compound is according to a method of treating cancer in a patient comprising administering a pharmaceutically effective amount of a compound as defined in formula I: 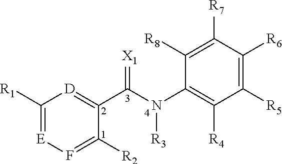
or derivative thereof, wherein
D is N or CR⁹; E is N or C¹⁰; F is N or CR¹¹;
R¹ is H, halide, OR¹², SR¹³, NR¹⁴, R¹⁵, 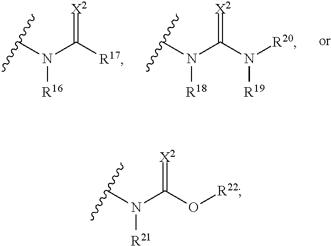
R² is H, OH or OR¹²;
R³ is H C₁₋₇ alkyl, C₂₋₇ alkenyl, C₂₋₇ alkynyl, C₂₋₆ heterocyclyl; C₆₋₁₂ aryl, C₇₋₁₄ alkaryl; C₃₋₁₀ alkheterocyclyl, or C₁₋₇ heteroalkyl; or R² and R³ combine to form a six-membered ring in which position 1 is connected to position 4 by 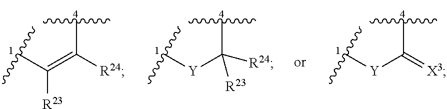
R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, 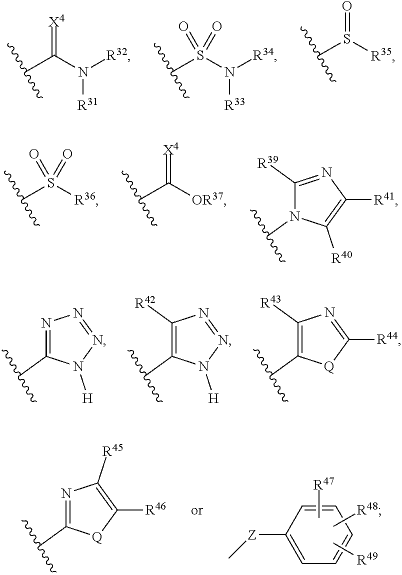
each X¹, X², X³, and X⁴ is, independently, O, S; or NR³⁸;
Y is CR²⁵R²⁶, O, S, or NR²⁷;
Z is O, S, or CR⁵⁰R⁵¹;
each Q is, independently, O, S, or NR⁵²;
R⁹, R¹⁰, and R¹¹ are each, independently, H, OH OR¹², C₁₋₇ alkyl, C₂₋₇ alkenyl, C₂₋₇ alkynyl, C₁₋₇ heteroalkyl, halide, or NO₂;
R¹² and R¹³ are each, independently, acyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₇ heteroalkyl;
R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, wherein the cancer comprises p53-deficient cells or cells wherein p53 is non-functional.

33. (canceled)

34. (canceled)

35. (canceled)
